(19) United States
(12) Patent Application Publication
(10) Pub. No.: US 2023/0148914 A1
(43) Pub. Date: May 18, 2023

(54) ANALYTE MEASURING DEVICE COMPRISING AN ADHESIVE PATCH

(71) Applicant: PKvitality, Paris (FR)

(72) Inventors: Luc PIERART, VILLEJUIF (FR); Anh-Minh LE, NEUILLY SUR SEINE (FR); Kevin Ly HAK, VILLIERS SUR MARNE (FR)

(73) Assignee: PKvitality, Paris (FR)

(21) Appl. No.: 17/917,123

(22) PCT Filed: Apr. 6, 2021
(86) PCT No.: PCT/FR2021/050598

§ 371 Date: Oct. 5, 2022

(30) Foreign Application Priority Data
Apr. 6, 2020 (FR) FR2003419

(51) Int. Cl. A61B 5/145; A61B 5/00

(52) U.S. Cl. CPC A61B 5/14514; A61B 5/685; A61B 5/68335; A61B 2560/0412; A61B 2560/0443; A61B 2562/162

(57) ABSTRACT

The present invention relates to a body monitoring system comprising a patch, the patch comprising a main body, the main body comprising a second face suitable for facing the skin of a user, the patch comprising an adhesive layer at least partially covering the second face, and a peelable cover covering the adhesive layer, the peelable cover comprising at least two separate portions, the portions being adjacent to each other, the main body and the adhesive layer comprise a central portion forming an opening suitable for receiving the capsule, the opening having a maximum diameter greater than 2 mm, preferably greater than 5 mm.

### FIELD OF THE NVENTION

The present invention relates to a body monitoring system via analysis of body, typically interstitial, fluid using microneedles. More specifically, the present invention relates to a patch for managing the holding of the microneedles in the skin.

### STATE OF THE ART

Some pathologies such as diabetes require daily monitoring of biochemical parameters of the human body, i.e. concentrations of some compounds (glycemia in the example of glucose).

For that, it is common practice to prick a point in the skin so as to form a droplet of blood, and to analyze this drop either reactively (for example with a strip), or electronically (for example by at least one analytical sensor), so as to estimate the target parameter(s).

Much less invasive advanced systems that simply analyze the interstitial fluid, that is to say the fluid that fills the space between the blood capillaries and the cells, are known. It has indeed an ionic composition close to that of blood plasma.

These advanced systems thus allow monitoring the desired biochemical parameters transcutaneously, that is to say without the need to regularly pierce the skin and take samples.

Devices with microneedles which have the advantage of being less invasive than conventional needles are known. However, it is important that these microneedles remain in place.

There are for that purpose indwelling devices where microneedles are held to the skin with an adhesive tape. However, it is desired to be able to carry out a continuous or quasi-continuous monitoring, which requires autonomous devices. The GlucoWatch device, which used iontophoresis (and not needles) can thus be mentioned.

The device of document WO2018104647 is also known, which has a casing comprising a removable capsule, the capsule accommodating microneedles configured to sample interstitial fluid. The casing for its part accommodates most part of the electronics.

This portable device, typically on the wrist, allows a continuous measurement and it suffices to change the capsule to change microneedles.

However, when such a device is worn on the wrist, a difficulty lies in the prior adjustment of the device to a comfortable place for the user. Indeed, the adjustment of the device is hardly compatible with the presence of microneedles in the skin because the microneedles can damage the skin during their insertion.

Devices of the prior art comprise a patch, but no adjustment means on a limb of the user. Such devices are also often applied to the skin in a single grip with an applicator, without the possibility of readjustment.

### DISCLOSURE OF THE INVENTION

One aim of the invention is to propose a solution for manufacturing a body surveillance system that allows solving at least partly the problems of the aforementioned prior art.

This aim is achieved in the context of the present invention thanks to a body monitoring system, comprising:
a capsule for measuring a body analyte, the capsule comprising a first face capable of being brought into contact with the skin of a user, and microneedles fixedly mounted on the first face,
a patch comprising a main body, the main body comprising a second face capable of being against the skin of a user, the patch comprising a layer of adhesive covering at least partly the second face, and a peelable cover covering the layer of adhesive,
the peelable cover comprising at least two separate parts, the parts being adjacent to each other,
the main body and the layer of adhesive comprising a central part in which an opening capable of receiving the capsule is formed, the opening having a maximum diameter greater than 2 mm, preferably greater than 5 mm,
the peelable cover entirely covering the opening,
the capsule being removably received in the opening, and
the peelable cover covering the microneedles.

The invention is advantageously completed by the following characteristics, taken individually or in any one of their technically possible combinations:
the patch extends mainly along a longitudinal direction, and the two parts are arranged on either side of an axis parallel to the longitudinal direction,
the patch has an oblong shape along the longitudinal direction,
each part of the peelable cover comprises a tab adapted to be gripped by a user,
at least one of the tabs is linked to one end of the peelable cover relative to the longitudinal direction,
the tab is fixedly mounted at one end of the peelable cover,
at least one tab mainly extends along a main axis having a non-zero component along the longitudinal direction,
the absolute value of an angle formed between the longitudinal direction and the main axis is preferably comprised between 30° and 60°,
the main body has a greater thickness than a thickness of the layer of adhesive, and preferably a thickness greater than 50 µm, in particular greater than 100 µm,
the patch has a contour, and parts of the contour arranged on either side of the opening along a direction transverse to the longitudinal direction form sawteeth,
the peelable cover comprises at least one external layer disposed opposite the main body relative to the rest of the peelable cover, the external layer covers the microneedles, the peelable cover also comprising at least one spacer arranged at least partly around the opening, a thickness of the spacer being configured to raise a part of the external layer covering the opening relative to the rest of the external layer so as to form a space between the external layer and a top of each microneedle,
the system comprises two spacers arranged on either side of the opening along a direction transverse to the longitudinal direction, each spacer extending mainly along the longitudinal direction.

Another aspect of the invention is a device intended to be attached to a limb, comprising:
a system in accordance with the invention,
a casing able to be removably fixed with the capsule of the system, inside which there is a battery and a processor, the processor being configured to process data obtained using the measurement made by at least one of the microneedles,
a strap configured to hold the casing in place on the limb.

### DESCRIPTION OF THE FIGURES

Other characteristics, aims and advantages of the invention will emerge from the following description, which is purely illustrative and not limiting, and which should be read in relation to the appended drawings in which:

FIG. 1 schematically illustrates a side view of a patch according to one embodiment of the invention.

FIG. 2 schematically illustrates a bottom view of a patch according to one embodiment of the invention.

FIG. 3 schematically illustrates a sectional view of a body monitoring system comprising a capsule and a patch according to one embodiment of the invention.

FIG. 4 schematically illustrates a bottom view of a patch comprising tabs according to one embodiment of the invention.

FIG. 5 schematically illustrates a bottom view of a patch having sawtooth contours according to one embodiment of the invention.

FIG. 6 schematically illustrates a section of a body monitoring system according to one embodiment of the invention, in which the patch comprises a spacer.

FIG. 7 schematically illustrates an exploded view of different layers forming a patch according to one embodiment of the invention.

FIG. 8 schematically illustrates an exploded view of different layers forming a patch according to one embodiment of the invention.

In all the figures, similar elements bear identical references.

### DEFINITIONS

It is meant by “diameter” of an opening the maximum size of this opening, regardless of the shape of the opening, namely the maximum distance between two points of a contour of the opening.

### DETAILED DESCRIPTION OF THE INVENTION

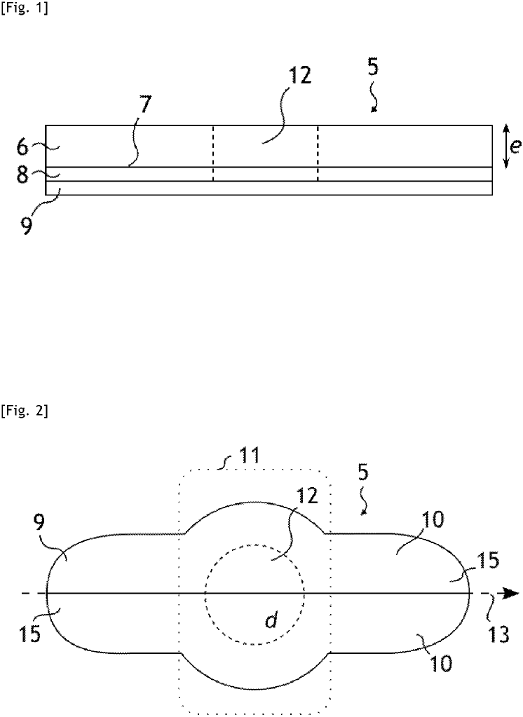

With reference to FIG. 1 and FIG. 2, a patch 5 comprises a main body 6. The main body 6 has an overall planar shape, and has a thickness e. The main body 6 comprises a second face 7 capable of being against the skin of a user when the body monitoring system 1 is worn on a limb of the user. The main body 5 can be made of polyurethane (PU). The patch 5 also comprises a first layer of adhesive 8 covering at least partly, preferably totally, the second face 7 of the main body 6.

The first layer of adhesive 8 preferably comprises an adhesive allowing the patch 5 to be reused on the skin of the user. The adhesive of the first layer of adhesive 8 may preferably comprise silicone.

The patch 5 also comprises a peelable cover 9 covering the first layer of adhesive 8. It is meant by “peelable cover” a surface coating which can be detached by a dry process and without breaking. In other words, the peelable cover 9 is intended to be detached from the first layer of adhesive 8 by the user, and allows it to be protected when it is covered thereby.

The main body 6 and the first layer of adhesive 8 comprise a central part 11. An opening 12 is formed in the central part 11. The opening 12 is intended to receive a capsule 2.

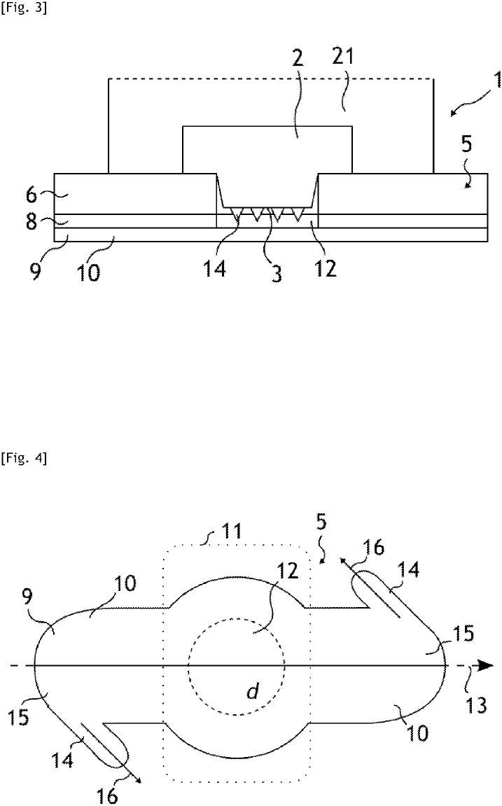

With reference to FIG. 3, the body detection system 1 comprises the patch 5 and a capsule 2 for measuring a body analyte. The capsule 2 comprises a first face 3, capable of being brought into contact with the skin of a user. Microneedles 4 are fixedly mounted on the first face 3. It is meant by “microneedle” a needle having a base and a top, and whose base has a size less than 600 µm, and whose distance from the base to the top of the needle is less than 1 mm, preferably less than 800 µm. The first face 3 is not limited to a planar face. It can have one or several recesses, in which the base of each microneedle 4 can be fixedly mounted.

The opening 12 of the patch 5 is configured to removably receive the capsule 2, as illustrated in FIG. 3. The opening 12 has a maximum diameter greater than 2 mm, in particular greater than 5 mm, and preferentially greater than 1 cm. Thus, the capsule 2 can be housed in the opening 12.

Referring to FIG. 2, the peelable cover 9 comprises at least two separate parts 10, each of the parts being adjacent to each other. The peelable cover 9 entirely covers both the opening 12 and the microneedles 4 of the capsule 2.

This configuration of the body monitoring system 1 allows both the removal of the peelable cover 9 after having disposed the system 1 against the skin of the user, without damaging the microneedles 4, by separating the peelable cover 9 into several separate parts, and the protection of the microneedles 4 from the external environment by covering the microneedles 4 with the peelable cover 9.

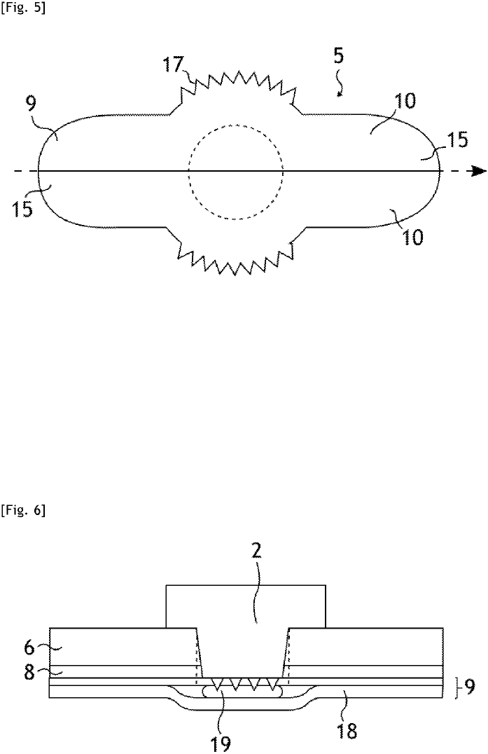

Referring to FIG. 2, FIG. 4 and FIG. 5, the patch 5 is preferably elongated along a longitudinal direction 13, or in other words extends mainly along a longitudinal direction 13. By “extends mainly along one direction”, it is meant that the maximum size of the patch is described by said direction. Thus, it is possible to increase the part of the patch 5 having a first layer of adhesive 8 and to increase the adhesion of the system 1 to the skin for a predetermined capsule size. The patch 5 preferably has an oblong shape along the longitudinal direction 13. Thus, the patch 5 does not form an angle likely to detach from the skin of the user.

Referring to FIG. 2, the patch 5 preferably comprises two parts 10, each part 10 being arranged on either side of an axis parallel to the longitudinal direction 13. Thus, once the system is disposed against the skin by the user, it is possible to detach each of the parts 10 from the rest of the patch 5 more easily and without damaging the microneedles 4 of the capsule 2.

As a variant, the patch 5 can also comprise four parts, each part being adjacent to two other parts, the parts 10 meeting in the center of the patch 5. The four parts 10 then form quarters of the surface of the patch 5. Thus, the removal of the peelable cover 9 when the system is disposed by the user against the skin is facilitated.

Referring to FIG. 4, at least part 10 of the peelable cover 9, and preferably each part 10 of the peelable cover 9 comprises a tab 14 adapted to be gripped by a user. This tab preferably differs from the shape of the rest of the patch 5. Thus, when the system 1 is placed against the skin of the user and adjusted to a suitable place for a measurement, the user can pull the tab 14 so as to remove the peelable cover from the rest of patch 5 without having to move the patch 5 to detach it.

The tab 14 can be manufactured by the following methods, in a non-limiting manner. The cutting shape of the peelable cover 9 can be made so as to form the tab 14 in the same plane and the same material as part of the rest of the peelable cover, in a single piece. As a variant, the tab 14 can be glued or fixed to part of the peelable cover. The layout of the tab 14 can be monitored by bending the tab.

When the patch 5 has an elongated shape, it has two ends 15. The tab is preferably linked, or fixedly mounted, to the end 15 of the patch 5. Thus, it is possible to remove the peelable cover 9 from the rest of the patch 5 by pulling the tab 14 and bringing it back towards the center of the system 1.

The tab 14 preferably extends along a main axis 16, the main axis having a zero component along the longitudinal direction 13. Thus, when the tab 14 is pulled by a user, the detachment of the peelable cover at its end 15 is favored, and the detachment of the peelable cover 14 from the rest of the patch 5 is thus facilitated. The absolute value of an angle formed between the longitudinal direction 13 and the main axis 16 is preferably comprised between 30° and 60°.

Referring to FIG. 1, the main body 6 has a greater thickness e than a thickness of the first layer of adhesive 8, and preferably a thickness greater than 50 µm, in particular greater than 100 µm. The thickness e of the main body 6 is in particular greater than the thickness of any other layer forming the patch 5. Thus, the rigidity of the patch 5 is ensured when the peelable cover is removed by the user.

Referring to FIG. 5, the patch 5 has a contour 17. The patch 5 can comprise parts of the contour 17, arranged on either side of the opening 12 along a direction transverse to the longitudinal direction 13, forming Sawteeth. The inventors have indeed discovered, by comparing two patches 5 having the same adhesive power to the skin of a user, that a patch 5 having parts of its sawtooth contour causes less pain when detaching the patch from the skin of the user than a patch that does not have this type of contour. Indeed, the sawteeth allow reducing the surface contact with the skin and therefore the pulling of the user’s hairs. It is meant by “sawteeth” that the contour 17 of the patch 5 has a notched shape. Preferably, the notches of the notched shape have a maximum length of 5 mm. Each notch may have a rounded top. The angle formed by two successive notches is preferably comprised between 20° and 70°.

Referring to FIG. 6, the peelable cover 9 preferably comprises at least one external layer 18 disposed opposite the main body 6 relative to the rest of the peelable cover 9. The external layer 18 covers at least the opening 12 and the microneedles 4. The peelable cover 9 also comprises at least one spacer 19 arranged at least partly around the opening 12. The spacer 19 has a thickness configured to raise part of the external layer 18 covering the opening 12 relative to the rest of the external layer 18, so as to form a space between the external layer 18 and the top of each microneedle 4. This configuration allows covering the microneedles 4 so as to protect them before using the system, while making it possible to align the first layer of adhesive 8 with the first face 3. Indeed, when using the system 1, these two faces are in contact with the skin and are preferably aligned in the system. However, in the absence of a spacer 19, the height of the microneedles 4 could exceed a surface of the first layer of adhesive 8 and be in contact with the peelable cover 9, which would risk damaging the microneedles 4. The space imposed by the spacer 19 between the external layer 18 and the top of each microneedle 4 is preferably greater than 10 µm, in particular 100 µm, and more preferably 500 µm.

The different elements of the peelable cover 9 can themselves be divided into several parts, so as to form the different parts 10 of the peelable cover 9. For example, the external layer 18 is preferably divided into two or more parts adjacent to each other. Thus, the system 1 preferably comprises several spacers 19, in particular two spacers 19, arranged on either side of the opening 12. In this configuration, each part 10 of the peelable cover 9 comprises a spacer 19. Thus, when removing the peelable cover 9 during the use of the system 1, the spacer 19 does not risk damaging the microneedles 4.

Referring to FIG. 7, the peelable cover 9 preferably comprises, against the first layer of adhesive 8, a first coating 701, intended to be in contact with the first layer of adhesive 8. The first coating 701 can be made of polyurethane, polyethylene or any material known from the prior art.

The first coating 701 is covered, on the side opposite that of the first layer of adhesive 8, by a third layer of adhesive 704. The third layer of adhesive 704 allows fixedly mounting a spacer body 703 on the first coating 701.

The spacer body 703 covers the third layer of adhesive 704 on the opposite side to the first coating 701. The spacer 19 preferably comprises the spacer body 703 and the third layer of adhesive 704. The spacer body 703 is preferably made of cotton.

A second layer of adhesive 702 covers the spacer body 703 on the opposite side to the third layer of adhesive 704.

A second layer of adhesive 702 can be arranged between the spacer body 703 and the external layer 18. The second layer of adhesive 702 preferably comprises an adhesive called strong adhesive, such as acrylic. The second layer of adhesive 702 allows attaching the external layer 18 to the first coating 701 and preferably at the same time maintaining the spacer 19 between the external layer 18 and the first coating 701.

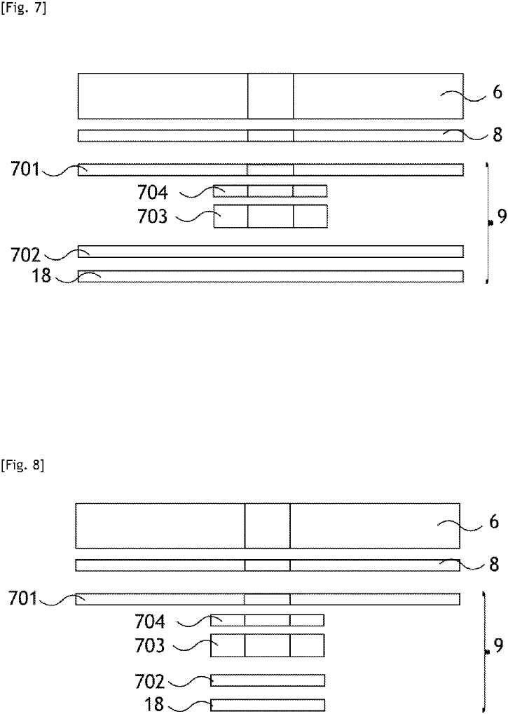

Referring to FIG. 8, the second layer of adhesive 702 and the external layer 18 cover only the spacer(s) 19, without being in contact with the first coating 701.

Another aspect of the invention is a device intended to be attached to a limb comprising a system according to one embodiment of the invention, a casing 21 able to be removably fixed with the capsule 2, inside which there is a battery and a processor. The processor is configured to process data obtained using the measurement made by at least one of the microneedles 4. The device also comprises a strap configured to hold the casing 21 in place on the limb. Thus, it is possible for the user, initially, to place the system 1 above his skin, so that the microneedles 4 are placed above a part of the skin suitable for coming into contact with the microneedles 4, then adjusting the position of the system 1, then removing the peelable cover 9 from the rest of the system 1, then finishing the tightening of the strap to secure the system to the limb of the user.

In general, the system thus allows protecting the microneedles 4 from possible contamination with the environment before the contact of the microneedles 4 with the skin of the user, and readjusting the device on a limb of the user in a more comfortable position, without fear of damaging the skin or the microneedles.

The system 1 is preferably adapted to measure a concentration of a body analyte chosen from glucose and lactate in the interstitial fluid of a user, and preferably continuously.

1. A body monitoring system, comprising:
a capsule for measuring a body analyte, the capsule comprising a first face capable of being brought into contact with the skin of a user, and microneedles fixedly mounted on the first face,
a patch comprising a main body, the main body (6) comprising a second face capable of being brought into contact the skin of a user, the patch comprising a first layer of adhesive covering at least partly the second face, and a peelable cover covering the layer of adhesive, characterized in that:
the peelable cover comprises at least two separate parts, the parts being adj acent to each other,
the main body and the first layer of adhesive comprise a central part in which an opening capable of receiving the capsule is formed, the opening having a maximum diameter greater than 2 mm, preferably greater than 5 mm,
the peelable cover entirely covers the opening,
the capsule is removably received in the opening, and
the peelable cover covers the microneedles.

2. The system according to claim 1, wherein the patch extends along a longitudinal direction, and wherein the two parts are arranged on either side of an axis parallel to the longitudinal direction.

3. The system according to claim 1, wherein the patch has an oblong shape along the longitudinal direction.

4. The system according to claim 1, wherein each part of the peelable cover comprises a tab adapted to be gripped by a user.

5. The system according to claim 4, wherein at least one of the tabs is linked to one end of the peelable cover relative to the longitudinal direction.

6. The system according to claim 4, wherein the tab is fixedly mounted at one end of the peelable cover.

7. The system according to claim 5, wherein at least one tab mainly extends along a main axis having a non-zero component along the longitudinal direction.

8. The system according to claim 7, wherein the absolute value of an angle formed between the longitudinal direction and the main axis is preferably comprised between 30° and 60°.

9. The system according to claim 1, wherein the main body has a greater thickness than a thickness of the layer of adhesive, and preferably a thickness greater than 50 µm, in particular greater than 100 µm.

10. The system according to claim 1, wherein the patch has a contour, and wherein parts of the contour arranged on either side of the opening along a direction transverse to the longitudinal direction form sawteeth.

11. The system according to claim 1, wherein the peelable cover comprises at least one external layer disposed opposite the main body relative to the rest of the peelable cover, the external layer (18) covering the microneedles, the peelable cover also comprising at least one spacer arranged at least partly around the opening, a thickness of the spacer being configured to raise a part of the external layer covering the opening relative to the rest of the external layer so as to form a space between the external layer and an top of each microneedle.

12. The system according to claim 11, comprising two spacers arranged on either side of the opening along a direction transverse to the longitudinal direction, each spacer extending mainly along the longitudinal direction .

13. A device intended to be attached to a limb, comprising:
a system (1) of claim 1,
a casing able be removably fixed with the capsule inside which there is a battery and a processor, the processor being configured to process data obtained using the measurement made by at least one of the microneedles,
a strap configured to hold the casing in place on the limb.

14. The system according to claim 6, wherein at least one tab mainly extends along a main axis having a non-zero component along the longitudinal direction.
